(19) United States
(12) Patent Application Publication
(10) Pub. No.: US 2006/0135446 A1
(43) Pub. Date: Jun. 22, 2006

(54) Composition for improving blood fluidity

(75) Inventors: Kinya Takagaki, Fukuoka (JP); Takeshi Mitsui, Fukuoka (JP)

Correspondence Address: AMIN & TUROCY, LLP, 1900 EAST 9TH STREET, NATIONAL CITY CENTER, 24TH FLOOR,, CLEVELAND, OH, 44114, US

(73) Assignee: Toyo Shinyaku Co., Ltd.

(21) Appl. No.: 11/022,438

(22) Filed: Dec. 22, 2004

(51) Int. Cl. A61K 31/7048; A61K 31/355; A61K 31/353; A61K 31/12; A61K 31/015

(52) U.S. Cl. 514/27; 514/456; 514/458; 514/763; 514/690

(57) ABSTRACT

The present invention provides a composition for improving blood fluidity that comprises proanthocyanidins and an antioxidant (which is a substance other than proanthocyanidins, ascorbic acid and its derivatives) as active components. The composition for improving blood fluidity of the present invention improves blood flow in the body and further improves the fluidity of blood.

### BACKGROUND OF THE INVENTION

1. Field of the Invention

The present invention relates to a composition for improving blood fluidity. This composition may be a food composition or pharmaceutical composition for improving fluidity of blood.

2. Description of the Related Art

In recent years, diseases associated with blood and circulatory system, such as arteriosclerosis and cerebral infarction, and diseases that have a harmful effect on blood circulation, such as hyperlipemia and diabetes, have been increasing because of changes in life environment including westernization of eating habits, lack of exercise, and excessive stress. These diseases cause a reduced blood flow in microvessels and capillary vessels, and thus it is pointed out that they may have various harmful effects on the body. Moreover, it is also pointed out that blood flow is related to itchy skin, fatigue, and hypertension, for example.

Generally, circulation of blood, that is, blood flow is reduced under the influence of (1) deterioration in the fluidity of blood due to hyperlipidemia and hyperglycemia; (2) reduction in the blood cell fluidity, that is, reduction in the flexibility of erythrocytes and leukocytes or increase in their viscosity; (3) increase in the platelet aggregation ability; and the like. In particular, blood cells, such as erythrocytes and leukocytes, are said to constitute about 40% of blood by volume, and have an effect especially on the fluidity of blood in micro blood vessels. If such a state in which blood flow is reduced continues for a long period of time, then, for example, the flexibility of blood vessels is lost, the flexibility of erythrocytes deteriorates, microvessels become more likely to be clogged with erythrocytes or leukocytes, or a blood clot is more easily formed. All of these phenomena contribute to the development of the circulatory system diseases as described above. In critical cases, the flow of blood may be blocked, resulting in necrosis of tissue in the area of that blockage. Therefore, “flowability of blood in the body” is regarded as important in maintaining good health.

Before now, a large number of foods and food components that may improve blood flow have been reported. Examples of familiar foodstuffs include black vinegar and umeboshi (pickled Japanese plum). Moreover, Japanese Laid-Open Patent Publication No. 7-138168 reports that a polar solvent extract of fish bile improves the fluidity of blood. Furthermore, Japanese Laid-Open Patent Publication No. 2002-97143 reports that glucosamine salts and glucosamine derivatives prevent blood clot formation or improve the fluidity of blood. However, the effects of improving blood flow that are provided by the above-mentioned foods and the like are mainly based on their abilities that are associated with the fluidity of blood, and these abilities are not sufficient. Furthermore, there is no consideration on whether these abilities that are associated with the fluidity of blood actually improve blood flow in the body.

Therefore, there is a demand for a composition for improving blood flow that improves blood flow in the body in the true sense, in other words, that improves the fluidity of blood and also has an excellent effect of protecting blood vessels.

### SUMMARY OF THE INVENTION

As a result of in-depth research on compositions for improving the fluidity of blood in the body, the inventors of the present invention found that a composition containing proanthocyanidins and an antioxidant as active components has excellent abilities of improving blood fluidity and protecting blood vessels, and found that the improvement of blood fluidity is achieved by improving the fluidity of blood cells, such as erythrocytes and leukocytes.

The composition for improving blood fluidity of the present invention comprises a proanthocyanidin and an antioxidant as active components, wherein the antioxidant is a substance other than proanthocyanidins, ascorbic acid and its derivatives.

In a preferred embodiment, the antioxidant is at least one selected from the group consisting of vitamin P-like substances, carotenoids, tocopherol and its derivatives, curcumin and its derivatives, ubiquinones, and lignans.

In a further preferred embodiment, the proanthocyanidin comprises at least 20 wt % of OPC (oligomeric proanthocyanidin).

According to the present invention, when the composition containing proanthocyanidins and an antioxidant as active components is ingested, it is possible to achieve an excellent effect of improving blood fluidity that cannot be achieved when each of these components are ingested alone. Furthermore, it is also possible to achieve an effect of protecting blood vessels. The composition of the present invention can be used effectively for foods, drugs, quasi-drugs, cosmetics, and the like.

### DESCRIPTION OF THE PREFERRED EMBODIMENT

Hereinafter, the composition for improving blood fluidity of the present invention will be described. It should be noted that the following description is not limiting the present invention, and it is apparent to those skilled in the art that various alternations can be made within the scope of the spirit of the present invention.

The composition for improving blood flow of the present invention comprises proanthocyanidins and an antioxidant as active components, and the antioxidant is a substance other than proanthocyanidins, ascorbic acid and its derivatives. This composition for improving blood flow can comprise other components, if necessary. Hereinafter, these components will be described.

(Proanthocyanidins)

In the present invention, proanthocyanidins refer to a group of compounds that are condensation products having flavan-3-ol and/or flavan-3,4-diol as a constituent unit and having a degree of polymerization of 2 or more. Proanthocyanidins are known to have various activities such as an antioxidation ability.

In this specification, among proanthocyanidins, condensation products having a degree of polymerization of 2 to 4 are referred to as oligomeric proanthocyanidins (OPCs). OPCs, which are one type of polyphenol, are potent antioxidants produced by plants, and cannot be produced in the human body.

OPCs are contained concentratedly in portions of plant leaves, bark, or skin or seeds of fruits. More specifically, they are contained in the bark of pine, oak, bayberry, and the like; the fruit or seeds of grape, blueberry, raspberry, cranberry, strawberry, avocado, locust, cowberry, and the like; the hull of barley, wheat, soybean, black soybean, cacao, adzuki bean, and conker; the inner skin of peanuts; and the leaves of ginkgo, for example. Moreover, it is known that OPCs are also contained in cola nuts in West Africa, the roots of Rathania in Peru, and Japanese green tea.

Therefore, for the proanthocyanidins, food raw materials, such as ground products or extracts from the above-mentioned barks, fruits, or seeds that contain a large amount of OPCs, can be used. In particular, it is preferable to use a pine bark extract. Among proanthocyanidins, OPCs are especially abundant in pine bark, and thus, a pine bark extract is preferably used for a raw material of the proanthocyanidins in the present invention.

Hereinafter, a method for preparing proanthocyanidins will be described taking a pine bark extract that contains OPCs abundantly as an example.

As the pine bark extract, an extract from the bark of plant belonging to Pinales, such as French maritime pine (Pinus martima), Larix leptolepis, Pinus thunbergii, Pinus densiflora, Pinus parviflora, Pinus pentaphylla, Pinus koraiensis, Pinus pumila, Pinus luchuensis, utsukushimatsu (Pinus densiflora form. umbraculifera), Pinus palustris, Pinus bungeana, and Anneda in Quebec, Canada, can be preferably used. Among these, French maritime pine (Pinus martima) bark extract is preferable.

French maritime pine refers to maritime pines that grow in a part of the Atlantic coastal area in southern France. It is known that the bark of this French maritime pine contains proanthocyanidins, organic acids, and other bioactive substances, and proanthocyanidins, which are the main component of the French maritime pine bark, are known to have a potent antioxidation ability of removing active oxygen.

The pine bark extract is obtained by extracting the bark of the above-mentioned pines using water or an organic solvent. When water is used, it is preferable to employ warm water or hot water. In order to increase the extraction efficiency, it is preferable to add a salt such as sodium chloride to the water. As the organic solvent that can be employed for extraction, an organic solvent that is acceptable for production of foods or pharmaceuticals can be employed. Examples of such solvent include methanol, ethanol, 1-propanol, 2-prbpanol, 1-butanol, 2-butanol, acetone, hexane, cyclohexane, propylene glycol, aqueous ethanol, aqueous propylene glycol, methyl ethyl ketone, glycerin, methyl acetate, ethyl acetate, diethyl ether, dichloromethane, edible oils or fats, 1,1,1,2-tetrafluoroethane, and 1,1,2-trichloroethene. The water and the organic solvents may be used alone or in combination. In particular, water, hot water, ethanol, aqueous ethanol, and aqueous propylene glycol are preferably used.

The method for extracting proanthocyanidins from pine bark is not particularly limited, and heat extraction or supercritical fluid extraction can be employed, for example.

Supercritical fluid extraction is a method for performing extraction using a supercritical fluid. A supercritical fluid is in a state that is above the liquid-vapor critical point in the phase diagram showing critical temperature and critical pressure. Examples of compounds that can be employed as a supercritical fluid include carbon dioxide, ethylene, propane, and nitrous oxide (laughter gas). Carbon dioxide is preferably used.

Supercritical fluid extraction includes an extraction step in which a target component is extracted with a supercritical fluid and a separation step in which the target component is separated from the supercritical fluid. In the separation step, any separation process can be employed, examples of which include a separation based on a change in pressure, a separation based on a change in temperature, and a separation using an adsorbent or absorbent.

Moreover, it is also possible to perform supercritical fluid extraction in which an entrainer is added. In this method, extraction is performed using an extracting fluid obtained by adding, for example, ethanol, propanol, n-hexane, acetone, toluene, or another aliphatic lower alcohol, aliphatic hydrocarbon, aromatic hydrocarbon, or ketone at about 2 to 20 W/V% to a supercritical fluid, so that the solubility of a target substance to be extracted, such as OPCs and catechins (described later), in the extracting fluid is dramatically increased or the selectivity of separation is enhanced. Thus, a pine bark extract is obtained efficiently.

Since supercritical fluid extraction can be performed at a relatively low temperature, it has the following advantages: it is applicable for extracting substances that deteriorate or decompose at high temperatures; the extracting fluid does not remain; and the extracting fluid can be recovered and recycled, so that a step of removing the extracting fluid and the like can be omitted, and thus, the process can be simplified.

Furthermore, methods other than those mentioned above can be employed for extraction from pine bark, and the examples of which include a batch method using liquid carbon dioxide, a reflux method using liquid carbon dioxide, a reflux method using supercritical carbon dioxide, and the like.

It is also possible to employ a combination of a plurality of extraction processes to perform extraction from pine bark. By combining a plurality of extraction processes, pine bark extracts with various components can be obtained.

In the present invention, the pine bark extract that contains proanthocyanidins as the main component is specifically prepared using the following method. However, this method is merely an example, and the pine bark extract used for the present invention is not limited to the extract obtained by this method.

First, 1 kg of the bark of French maritime pine is immersed in 3 L of a saturated solution of sodium chloride, and extraction is performed for 30 minutes at 100° C. to obtain an extract liquid (extraction step). Then, the extract liquid is filtrated, and the resultant insoluble material is washed with 500 ml of a saturated solution of sodium chloride to obtain a washed liquid (washing step). The extract liquid and the washed liquid are combined to obtain a crude extract liquid of pine bark.

Next, 250 ml of ethyl acetate is added to this crude extract liquid, mixed, and separated to obtain an ethyl acetate layer. This process is repeated five times, and the obtained ethyl acetate layers are combined. The resultant ethyl acetate extract is added directly to 200 g of anhydrous sodium sulfate for drying. Then, this ethyl acetate extract is filtrated, and the filtrated extract is concentrated under a reduced pressure to a volume of ⅕ of the original filtrated extract. The concentrated ethyl acetate extract is poured into 2 L of chloroform and stirred, and the resultant precipitate is recovered by filtration. Subsequently, this precipitate is dissolved in 100 ml of ethyl acetate, and then the resultant solution is added to 1 L of chloroform to form a precipitate. This process is repeated twice, and thus, a washing process is accomplished. With this method, for example, about 5 g of pine bark extract containing at least 20 wt % of OPCs and at least 5 wt % of catechins can be obtained.

In view of the safety when used in foods or drugs, it is preferable to extract proanthocyanidins from pine bark using ethanol, water, or the like, more preferably while heating, and purify the extract using an adsorption resin (e.g., DIAION HP-20, Sephadex-LH20, and chitin) or the like, or an ultrafiltration membrane. In the present invention, a pine bark extract having a high proanthocyanidin content that is obtained through such a process is preferably used.

In the composition for improving blood fluidity of the present invention, extracts from the above-mentioned raw material plants that contain proanthocyanidins are preferably employed. As described above, pine bark extracts contain a large amount of proanthocyanidins, and among these, pine bark extracts that contain a large amount of proanthocyanidins (condensation products) having a lower degree of polymerization are preferably used. As such condensation products, condensation products having a degree of polymerization of 2 to 30 (dimer to 30-mer) are preferable, condensation products having a degree of polymerization of 2 to 10 (dimer to decamer) are more preferable, and condensation products having a degree of polymerization of 2 to 4 (dimer to tetramer; i.e., OPCs) are even more preferable.

In the present invention, proanthocyanidins containing at least 20 wt % of OPCs are preferably used. More preferably, the OPC content is at least 30 wt %. As an extract containing such proanthocyanidins, a pine bark extract is preferably used.

When proanthocyanidins having a high OPC content are used, a better effect of improving blood fluidity can be achieved than in the case where proanthocyanidins having a high degree of polymerization (having a low OPC content) are used.

There is no particular limitation regarding the proanthocyanidin content in a plant (bark) extract, but it is preferable that the proanthocyanidin content in an extract is less than 80 wt % and preferably less than 75 wt % because the bioactivity of the proanthocyanidins themselves may be lowered when the proanthocyanidin content in the plant (bark) extract is high.

Since OPCs are antioxidants as described above, they also provide an effect of reducing the possibility of adult diseases, such as cancer and cardiac diseases, an effect of improving allergic diathesis, such as arthritis, atopic dermatitis, and pollenosis, an effect of inhibiting oxidation and degradation of collagen, and the like.

Furthermore, it is known that in addition to the antioxidation ability, OPCs also provide an effect of recovering the strength and elasticity of blood vessels, an effect of decreasing cholesterol and LDL in blood, an effect of decreasing blood pressure with respect to hypertension, an effect of preventing adhesion of cholesterol, an effect of regenerating vitamin E that has been degraded by active oxygen, an effect of serving as an enhancer of vitamin E, and the like.

In particular, by virtue of the antioxidation ability, the effect of decreasing cholesterol in blood, the effect of decreasing blood pressure with respect to hypertension, the effect of recovering the elasticity of blood vessels, and the effect of preventing adhesion of cholesterol, blood vessels can be protected and also the fluidity of blood can be improved, so that blood flow in the body can be improved synergistically.

Furthermore, OPCs can improve blood flow by maintaining the fluidity of blood cells. It is known that a reduction in the fluidity of erythrocytes or leukocytes leads to a reduction in blood fluidity especially in microvessels. The reduction in this flowability is caused by chemical or physical stimulation, such as an increase in the viscosity of blood cells due to stress of oxidation, inflammation, or the like, a change in blood pressure, and vascular constriction. Regarding erythrocytes, it has already been found that the fluidity of erythrocytes is reduced when such chemical or physical stimulation, that is, an extrinsic signal is transmitted to the inside of erythrocytes to cause a biochemical change. Since OPCs have properties of enhancing the antioxidation effect and enhancing the strength and elasticity of blood vessels as described above, such chemical or physical stimulation to erythrocytes and leukocytes can be reduced by OPCs. Thus the blood cell fluidity is maintained, and therefore, blood flow can be improved.

The above-mentioned plant extracts contain catechins as well as proantocyanidins, in particular, OPCs. The term “catechins” is a general term referring to polyhydroxyflavan-3-ols. As the catechins, for example, (+)-catechin (which is referred to as “catechin” in a narrow sense), (−)-epicatechin, (+)-gallocatechin, (−)-epigallocatechin, epigallocatechin gallate, epicatechin gallate, and afzelechin are known. Gallocatechin, afzelechin, 3-galloyl derivatives of (+)-catechin, and 3-galloyl derivatives of gallocatechin are isolated from natural products, in addition to the above-mentioned (+)-catechin. Catechins are known to have a cancer inhibiting ability, an arteriosclerosis preventing ability, a lipid metabolism disorder inhibiting ability, a blood pressure elevation inhibiting ability, a platelet aggregation inhibiting ability, an antiallergic ability, an antiviral ability, an antibacterial ability, a dental caries preventing ability, a halitosis preventing ability, an intestinal flora normalization ability, an active oxygen or free radical eliminating ability, an antioxidation ability, and the like. Moreover, catechins are known to have an antidiabetic ability of inhibiting an elevation of blood glucose. Catechins alone have poor solubility in water and exhibit low bioactivity, but the solubility in water is increased and the catechins are activated in the presence of OPCs. In this way, catechins work effectively when ingested together with OPCs.

It is preferable that catechins are contained in the above-mentioned raw material plant extracts in a ratio of 5 wt % or more. More preferably, a formulation is prepared so that it contains a raw material plant extract containing at least 20 wt % of OPCs and furthermore, contains catechins in a ratio of 5 wt % or more. For example, when the catechin content in an extract is less than 5 wt %, it is possible to add catechins so that the final catechin content becomes at least 5 wt %. It is most preferable to use a pine bark extract containing at least 20 wt % of OPCs and at least 5 wt % of catechins.

(Antioxidants)

The antioxidant used in the present invention is a substance having an antioxidation ability or a reduction ability, and is a substance other than proanthocyanidins, ascorbic acid and its derivatives. Plant materials containing such substance and extracts derived from plants or microorganisms containing such substance also can be used. The antioxidant can promote the effect of proanthocyanidins, in particular, OPCs. Namely, the antioxidant can exhibit the effect of improving blood fluidity more effectively.

Examples of the antioxidant include vitamin P-like substances (e.g., hesperidin, rutin, quercetin, and derivatives of quercetin); carotenoids (e.g., astaxanthin, lycopene, and β-carotene); vitamin B group substances; tocopherol and its derivatives (e.g., α-tocopherol and tocotrienol); ubiquinones (e.g., CoQ10); lignans (e.g, sesamin and sesamolin); curcumin and its derivatives; capsaicin and its analogues; gingenol analogues (contained in spices); diarylheptanoids; L-cysteine and derivatives thereof, and their salts; riboflavin; SOD; mannitol; tryptophan; histidine; gallic acid and its derivatives; and the like.

Examples of the plant materials containing the above-mentioned antioxidants and the extracts derived from plants or microorganisms containing these substances include plants, such as tea plants, sesame, eucalyptus, tartary buckwheat, and spice plants (turmeric, capsicum, ginger, perilla, rosemary, nutmeg, cinnamon, clove, sage, and thyme); extracts from these plants; and extracts derived from microorganisms, such as glutathione yeast extract.

(Other Components)

The composition for improving blood fluidity of the present invention can further comprise other components that provide an effect of improving blood fluidity, if necessary. Examples of such components having an ability of improving the fluidity of blood include, but are not limited to, black vinegar and ume (Japanese plum) flesh, and their extracts; sulfur-containing organic compounds contained in onion or garlic, and their extracts; chitin and chitosan and their derivatives; glucosamine salts and their derivatives; vitamin K, vitamin D, and vitamin E; and water-soluble dietary fibers. In particular, sulfur-containing organic compounds, vitamin K, vitamin E, and chitin and chitosan and their derivatives can be preferably used in order to enhance the ability of suppressing blood glucose level, lipid level in blood, and high blood pressure; the antithrombotic ability; the ability of decreasing cholesterol in blood; and the like.

Moreover, when the composition for improving blood fluidity of the present invention is used in foods or pharmaceuticals, the composition may contain various components that are commonly used for foods or pharmaceuticals, such as excipients, extenders, binders, thickeners, emulsifiers, lubricants, humectants, suspending agents, coloring agents, flavors, nutritions, and food additives, as appropriate. Examples of the nutritions include royal jelly, vitamins, proteins, calcium substances such as eggshell calcium, lecithin, chlorella powder, Angelica keiskei powder, and molokheiya powder. Examples of the food additives include stevia powder, ground green tea powder, lemon powder, honey, maltitol, lactose, and sugar solutions.

(Composition for Improving Blood Fluidity)

The composition for improving blood fluidity of the present invention contains the above-mentioned proanthocyanidins and antioxidant as active components in any suitable ratio, and contains other components, if necessary. Preferably, the antioxidant can be contained in an amount of 0.01 to 5000 parts by weight and more preferably 0.05 to 2000 parts by weight with respect to 100 parts by weight of the proanthocyanidins.

There is no particular limitation regarding the proanthocyanidin content in the composition for improving blood fluidity of the present invention, but it is preferable that proanthocyanidins are contained in such an amount that the daily intake amount of the proanthocyanidins is 0.001 g to 0.2 g, preferably 0.002 g to 0.15 g, and more preferably 0.002 g to 0.08 g. In particular, in the case of a pine bark extract, its effects can be achieved even when the daily intake amount of proanthocyanidins is 0.001 g to 0.05 g. In this way, even when the amount of proanthocyanidins is relatively small, the effect of improving blood fluidity that is associated with improvement of the fluidity of blood cells such as erythrocytes and leukocytes can be achieved. Also, there is no limitation regarding the antioxidant content, but it is preferable that an antioxidant is contained in the composition in such an amount that the daily intake amount of the antioxidant satisfies the above-mentioned recommended amount for that antioxidant.

Furthermore, it is preferable that the proanthocyanidins in the composition of the present invention is contained in the composition in a ratio of preferably 0.001 wt % to 50 wt % and more preferably 0.005 wt % to 20 wt %. In particular, when the composition is applied to the skin, the effect of improving blood fluidity can be achieved with a smaller amount because the composition is administered (applied) topically. In such case, it is preferable that the proanthocyanidins are contained in the composition in a ratio of preferably 0.0001 wt % to 10 wt % and more preferably about 0.001 wt % to 5 wt %.

The composition for improving blood fluidity can be made into various forms by combining the above-mentioned components using a method that is commonly employed by those skilled in the art. For example, the composition may be prepared in the form of tablets or pills, for example, by adding an excipient or the like to a pine bark extract containing proanthocyanidins and an antioxidant. Alternatively, the composition may be prepared in the form of powder or in other forms without being shaped. Examples of other forms include the forms of capsules such as hard capsules and soft capsules, powder, granule, tea bags, candy, liquid, and paste. Among these, a liquid form (e.g., beverage) is preferable.

When the composition for improving blood fluidity of the present invention is ingested, there is no limitation regarding the method for ingesting the composition. According to the form of the composition or according to preference, the composition may be eaten or drunk as it is, or may be dissolved in water, hot water, milk, or the like and drunk. Alternatively, a liquid containing the components of the composition obtained by percolation may be drunk.

The composition for improving blood fluidity of the present invention exhibits an excellent effect of improving blood flow by improving the fluidity of blood cells. Therefore, the composition does not affect the platelet aggregation ability, platelet count, blood plasma components such as cholesterol and neutral fat, erythrocyte count, leukocyte count, and the like. The composition of the present invention further improves the flexibility and the strength of blood vessels, so that an effect of improving blood flow in the body, in particular, an effect of improving peripheral blood flow can be achieved. Furthermore, the improvement of blood flow leads to improvement of the health of the entire body. In the composition of the present invention, among plant extracts containing proanthocyanidins, a pine bark extract that is obtained by extraction using water, in particular, hot water or ethanol is preferred because such an extract particularly provides excellent effects of improving the fluidity of blood cells and improving blood fluidity. The composition of the present invention can be used for foods, drugs, quasi-drugs, cosmetics, and the like.

### EXAMPLES

Hereinafter, the present invention will be described by way of examples. However, the present invention is not limited to these examples.

### Example 1

### Production of Food 1

A hot water extract of pine bark (produced by TOYO SHINYAKU Co., Ltd.) containing at least 40 wt % of proanthocyanidins (OPC content: at least 20 wt % in the extract) and at least 5 wt % of catechins, α-tocopherol, and an excipient (i.e., a mixture of crystalline cellulose, sucrose ester, silicon dioxide, and eggshell calcium) were mixed in a ratio (wt %) shown in Table 1, and tablets (about 250 mg per tablet) were produced from the resultant mixture. These tablets were referred to as “Food 1”.

### Examples 2 to 6

### Production of Foods 2 to 6

Tablets (about 250 mg per tablet) were produced from the mixture of the components shown in Table 1 in the same manner as in Example 1. These tablets were referred to as “Food 2”, “Food 3”, “Food 4”, “Food 5”, and “Food 6”, respectively.

### Comparative Examples 1 to 3

### Production of Foods 7 to 9

Tablets (about 250 mg per tablet) were produced from the mixture of the components shown in Table 1 in the same manner as in Example 1. These tablets were referred to as “Food 7”, “Food 8”, and “Food 9”, respectively.

TABLE 1

| | | Ex. | | | | | | Com. Ex. | | |
|---|---|---|---|---|---|---|---|---|---|---|
| | | 1 | 2 | 3 | 4 | 5 | 6 | 1 | 2 | 3 |
| Components | | Food 1 | Food 2 | Food 3 | Food 4 | Food 5 | Food 6 | Food 7 | Food 8 | Food 9 |
| With Pro*^{1a} | Pine bark extract | 10 | 10 | 10 | 10 | 10 | 10 | 10 | — | — |
| Without Pro*^{1b} | L-theanine*² | — | — | — | — | — | — | — | 10 | 10 |
| Antioxidants | α-Tocopherol | 0.1 | — | — | — | — | — | — | — | 0.1 |
| | CoQ10*³ | — | 0.1 | — | — | — | — | — | — | — |
| | Sesame extract*⁴ | — | — | 25 | — | — | — | — | — | — |
| | Hesperidin | — | — | — | 25 | — | — | — | — | — |
| | Dry powder of turmeric*⁵ | — | — | — | — | 5 | — | — | — | — |
| | Astaxanthin*⁶ | — | — | — | — | — | 0.1 | — | — | — |

Unit: wt %
The remaining part is excipient.
*^{1a}Proanthocyanidins are contained in this substance.
*^{1b}Proanthocyanidins are not contained in this substance.
*²Taiyo Kagaku Co., Ltd.
*³NISSHIN PHARMA INC.
*⁴Containing 0.9 wt % of lignans (TAKEMOTO OIL & FAT CO., LTD.)
*⁵Containing 0.9 wt % of curcumin (Japan Ukon Industry Corporation)
*⁶Maruzen Pharmaceuticals Co., Ltd.

### Example 7

### Measurement of Blood Passage Time of Human Blood

The blood passage time of human blood before and after the ingestion of a test food was measured in the following manner using each of the above-mentioned Foods 1 to 3 in the examples and Foods 7 to 9 in the comparative examples as the test food, and the effect of improving blood fluidity was evaluated. First, 30 healthy men between the ages of 22 and 63 served as subjects, and the subjects were divided into six groups randomly. The subjects in one group ingested one tablet of the Food 1 daily for two weeks. Similarly, the subjects in other groups ingested one tablet of the Food 2, 3, 7, 8, or 9. One tablet of each of the Foods 1, 2, 3, and 7 contained 25 mg of the pine bark extract. Blood samples were collected immediately before starting the ingestion of the foods and two weeks after the start of the ingestion. The blood samples were collected from the median cubital vein using a vacuum blood collection tube (manufactured by TERUMO CORPORATION: treated with heparin sodium) while the subjects were resting in a sitting position. The subjects did not have a breakfast on the days on which the blood samples were collected. The obtained blood (test blood) was immediately used for measurement of the blood passage time.

The blood passage time was measured using MC-FAN (manufactured by Hitachi Haramachi Electronics Co., Ltd.). As microfabricated channels serving as a blood vessel model through which blood flows, a silicon single crystal substrate (Bloody6-7; manufactured by Hitachi Haramachi Electronics Co., Ltd.) that is a substrate provided with a micro channel array having 8736 parallel micro grooves, each having a channel depth of 4.5 μm, a channel width (i.e., width of the channel at half depth of the channel) of 7 μm, and a channel length of 30 μm was used. Then, 100 μL of the blood were allowed to flow at a hydraulic pressure of 20 cm, and the passage time of the entire blood was measured as the blood passage time. Furthermore, the blood flow was filmed and recorded using a microscope-video camera system. For all measurement values, the average of the values obtained by three measurements was employed. The obtained blood passage time was corrected, taking the passage time required for 100 μL of physiological saline as 12 seconds. Table 2 shows the measurement results of the blood passage time. Each of the values in the table indicates the average value±standard error of the flow rate in each group.

TABLE 2

| | | | Blood passage time (sec.) | | |
|---|---|---|---|---|---|
| | | | Before | | Difference between |
| | | | ingestion | After ingestion of | before and after |
| | | Food | of food*¹ | food*¹ | ingestion of food |
| | | ingested | A | B | A − B |
| Ex. | 1 | Food 1 | 50.4 ± 2.1 | 42.8 ± 1.5 | 7.6 |
| | 2 | Food 2 | 49.8 ± 3.3 | 40.8 ± 1.2 | 9.0 |
| | 3 | Food 3 | 50.0 ± 1.9 | 41.5 ± 2.8 | 8.5 |
| Com. | 1 | Food 7 | 50.5 ± 2.4 | 45.9 ± 1.5 | 4.6 |
| Ex. | 2 | Food 8 | 50.1 ± 3.1 | 46.8 ± 1.9 | 3.3 |
| | 3 | Food 9 | 50.7 ± 2.0 | 46.1 ± 1.8 | 4.6 |

*¹average value ± standard error

Referring to the results in Table 2, the blood passage time in the groups in which the Food 1, 2, or 3 of the examples (Example 1, 2, or 3) was ingested was significantly shorter than that in the groups in which the Food 7, 8, or 9 of the comparative examples (Comparative Example 1, 2, or 3) was ingested. This shows that the proanthocyanidins and the antioxidant work synergistically to improve the fluidity of blood. Moreover, the longer the blood passage time of the subject before the ingestion was, the greater the effect of improving the fluidity of blood tended to be. The blood passage time in the group in which the Food 8 of Comparative Example 2 containing the theanine alone was ingested was equal to that in the group in which the Food 7 of Comparative Example 1 containing the proanthocyanidins alone was ingested. However, when comparing the Foods 8 and 9 (Comparative Examples 2 and 3), the combination of the theanine and the antioxidant could not achieve the synergistic effect of improving the fluidity of blood that was provided by the combination of the proanthocyanidins and any of the antioxidants (Foods 1 to 3) as described above.

### Example 8

### Evaluation of Effect of Improving Blood Flow

The following experiment was performed in order to confirm the effect of improving blood flow in the body. First, a total of 45 subjects were divided into groups of 5 each, and the blood flow rate of the subjects was measured before ingestion of the foods. Then, the subjects in one group ingested one tablet of the Food 1 daily for two weeks, and the blood flow rate was measured again after the end of the ingestion period. The blood flow rate was measured at a region under the right forearm skin using a rheometer (laser blood perfusion imager PIM II; Perimed AB, Sweden). For the Foods 2 to 9, the operation was performed in the same manner as described above, and the blood flow rate was measured before and after the ingestion of the foods. Table 3 shows the results. Each of the values in the table indicates the average value±standard error, and larger values indicate a higher blood flow rate.

TABLE 3

| | | | Blood flow rate | | |
|---|---|---|---|---|---|
| | | | Before | | Difference between |
| | | | ingestion | After ingestion | before and after |
| | | Food | of food | of food | ingestion of food |
| | | ingested | A | B | B − A |
| Ex. | 1 | Food 1 | 1.37 ± 0.05 | 1.55 ± 0.06 | 0.18 ± 0.05 |
| | 2 | Food 2 | 1.38 ± 0.04 | 1.59 ± 0.05 | 0.21 ± 0.04 |
| | 3 | Food 3 | 1.36 ± 0.03 | 1.56 ± 0.05 | 0.20 ± 0.04 |
| | 4 | Food 4 | 1.35 ± 0.06 | 1.6 ± 0.05 | 0.25 ± 0.05 |
| | 5 | Food 5 | 1.38 ± 0.02 | 1.58 ± 0.03 | 0.20 ± 0.03 |
| | 6 | Food 6 | 1.34 ± 0.04 | 1.55 ± 0.04 | 0.21 ± 0.04 |
| Com. | 1 | Food 7 | 1.37 ± 0.03 | 1.49 ± 0.04 | 0.12 ± 0.03 |
| Ex. | 2 | Food 8 | 1.35 ± 0.04 | 1.43 ± 0.04 | 0.08 ± 0.03 |
| | 3 | Food 9 | 1.38 ± 0.03 | 1.47 ± 0.05 | 0.09 ± 0.04 |

Each of the values indicates average value ± standard error.

Referring to the results in Table 3, the periphery blood flow rate was increased more in the groups in which the Food 1, 2, 3, 4, 5, or 6 of the examples (Example 1, 2, 3, 4, 5, or 6) was ingested than in the groups in which the Food 7, 8, or 9 of the comparative examples (Comparative Example 1, 2, or 3) was ingested. This shows that when a food containing proanthocyanidins and an antioxidant is ingested, an excellent effect of improving blood fluidity by which the blood flow rate in the tissues in the body is increased effectively can be achieved. When comparining the Foods 7, 8, and 9 of the comparative examples, it seems that the theanine provides an effect of improving the fluidity of blood that is equal to the effect of the proanthocyanidins (Foods 7 and 8). However, even when the theanine was combined with the antioxidant, the effect of improving blood flow that can be achieved by the combination of proanthocyanidins and the antioxidant of the present invention could not be achieved (Food 9).

### Example 9

### Production of Skin Lotion 1

Skin Lotion 1 was produced by mixing a hot water extract of pine bark (produced by TOYO SHINYAKU Co., Ltd.) containing at least 40 wt % of proanthocyanidins (OPC content: at least 20 wt % in the extract) and at least 5 wt % of catechins, α-tocopherol, and a base material according to the ratio shown in Table 4.

### Examples 10 to 14

### Production of Skin Lotions 2 to 6

Skin Lotions 2 to 6 were produced by mixing the components shown in Table 4 in the same manner as in Example 9.

### Comparative Examples 4 and 5

### Production of Skin Lotions 7 and 8

Skin Lotions 7 and 8 were produced by mixing the components shown in Table 4 in the same manner as in Example 9.

### Example 15

### Effect of Improving Blood Flow Achieved by Application

First, 40 healthy persons between the ages of 20 and 50 were divided into a total of eight groups of 5 each. Then, three marks each measuring 2.0 cm square were previously put on one forearm of each of the subjects, and the blood flow rate was measured in the areas of those marks using a rheometer. After the measurement, 0.1 ml of the Skin Lotion 1 were applied to the measurement areas of the subjects in one group. The blood flow rate was measured again two hours after the application. The difference in the blood flow rate between before and after the application was calculated, and the effect of improving blood flow achieved by the application was evaluated. For the Skin Lotions 2 to 8, the operations were performed in the same manner as described above, and the effect of improving blood flow achieved by the application of each skin lotion was evaluated. Table 5 shows the results.

TABLE 4

| | | Ex. | | | | | | Com. Ex. | |
|---|---|---|---|---|---|---|---|---|---|
| | | 9 | 10 | 11 | 12 | 13 | 14 | 4 | 5 |
| | | Skin | Skin | Skin | Skin | Skin | Skin | Skin | Skin |
| | | Lotion 1 | Lotion 2 | Lotion 3 | Lotion 4 | Lotion 5 | Lotion 6 | Lotion 7 | Lotion 8 |
| Pine bark extract | | 0.1 | 0.1 | 0.1 | 0.1 | 0.1 | 0.1 | 0.1 | — |
| (Proanthocyanidins) | | | | | | | | | |
| Antioxidants | α-Tocopherol | 0.01 | — | — | — | — | — | — | 0.1 |
| | CoQ10 | — | 0.01 | — | — | — | — | — | 0.1 |
| | Sesame extract | — | — | 0.01 | — | — | — | — | — |
| | Hesperidin | — | — | — | 0.01 | — | — | — | 0.1 |
| | Dry powder of turmeric | — | — | — | — | 0.01 | — | — | — |
| | Astaxanthin | — | — | — | — | — | 0.01 | — | — |
| Base material | Glycerin | 6 | 6 | 6 | 6 | 6 | 6 | 6 | 6 |
| | Propylene glycol | 4 | 4 | 4 | 4 | 4 | 4 | 4 | 4 |
| | Ethanol | 5 | 5 | 5 | 5 | 5 | 5 | 5 | 5 |

Unit: wt %
The remaining part is water.

TABLE 5

| | | | Blood flow rate | | |
|---|---|---|---|---|---|
| | | | | | Difference |
| | | | | | between |
| | | | | | before and after |
| | | | Before | After | application |
| | | Skin lotion | application | application | of skin lotion |
| | | applied | A | B | B − A |
| Ex. | 9 | Skin Lotion 1 | 1.17 ± 0.12 | 1.65 ± 0.21 | 0.48 ± 0.19 |
| | 10 | Skin Lotion 2 | 1.18 ± 0.17 | 1.69 ± 0.19 | 0.51 ± 0.17 |
| | 11 | Skin Lotion 3 | 1.16 ± 0.11 | 1.58 ± 0.17 | 0.42 ± 0.14 |
| | 12 | Skin Lotion 4 | 1.18 ± 0.20 | 1.69 ± 0.13 | 0.51 ± 0.15 |
| | 13 | Skin Lotion 5 | 1.15 ± 0.14 | 1.61 ± 0.15 | 0.46 ± 0.15 |
| | 14 | Skin Lotion 6 | 1.14 ± 0.15 | 1.54 ± 0.13 | 0.40 ± 0.15 |
| Com. | 4 | Skin Lotion 7 | 1.17 ± 0.16 | 1.50 ± 0.19 | 0.33 ± 0.16 |
| Ex. | 5 | Skin Lotion 8 | 1.15 ± 0.19 | 1.35 ± 0.16 | 0.20 ± 0.15 |

Average value ± standard error

Referring to the results in Table 5, the blood flow rate was increased more in the groups in which the Skin Lotion 1, 2, 3, 4, 5, or 6 of the examples (Example 9, 10, 11, 12, 13, or 14) containing the proanthocyanidins and the antioxidant was applied than in the groups of the comparative examples in which the Skin Lotion 7 (Comparative Example 4) containing the proanthocyanidins alone or the Skin Lotion 8 (Comparative Example 5) containing the antioxidant alone was applied. This shows that when a skin lotion containing proanthocyanidins and an antioxidant is applied, it is possible to achieve an excellent effect of improving blood flow that cannot be achieved when each of the components is contained alone.

The invention may be embodied in other forms without departing from the spirit or essential characteristics thereof. The embodiments disclosed in this specification are to be considered in all respects as illustrative and not limiting. The scope of the invention is indicated by the appended claims rather than by the foregoing description, and all changes which come within the meaning and range of equivalency of the claims are intended to be embraced therein.

1. A composition for improving blood fluidity, comprising a proanthocyanidin and an antioxidant as active components,
wherein the antioxidant is a substance other than proanthocyanidins, ascorbic acid and its derivatives.

2. The composition for improving blood fluidity of claim 1, wherein the antioxidant is at least one selected from the group consisting of vitamin P-like substances, carotenoids, tocopherol and its derivatives, curcumin and its derivatives, ubiquinones, and lignans.

3. The composition for improving blood fluidity of claim 1, wherein the proanthocyanidin comprises at least 20 wt % of OPC (oligomeric proanthocyanidin).

4. The composition for improving blood fluidity of claim 2, wherein the proanthocyanidin comprises at least 20 wt % of OPC (oligomeric proanthocyanidin).
